(19) United States
(12) Patent Application Publication
(10) Pub. No.: US 2003/0119685 A1
(43) Pub. Date: Jun. 26, 2003

(54) Personal cleansing compositions comprising silicone resin-containing adhesives

(75) Inventors: Michael Jahi Chappell, Ann Arbor, MI (US); Mannie Lee Clapp, Mason, OH (US); Eric Scott Johnson, Hamilton, OH (US); Rebecca Ann Taylor, Fairfield, OH (US); Colin Michael McHugh, Mason, OH (US); Edward Dewey Smith III, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/255,446

(22) Filed: Sep. 26, 2002

Related U.S. Application Data
(60) Provisional application No. 60/325,021, filed on Sep. 26, 2001.

(51) Int. Cl. A61K 7/075; A61K 7/50

(52) U.S. Cl. 510/130; 510/466; 510/119; 510/122

(57) ABSTRACT

Disclosed are personal cleansing compositions for rinse-off application to the hair, nails or skin. These compositions comprise a silicone-resin based adhesive including a silicone resin copolymer as a condensation product of an organosiloxane fluid and a silicone resin; a cleansing surfactant; and a carrier liquid. The personal cleansing compositions can also comprise as the silicone-resin based adhesive, in addition to or in place of the silicone resin copolymer, a combination of an organosiloxane resin and a diorganosiloxane fluid at a weight ratio of said resin to said fluid of from about 1:9 to about 10:1. The personal cleansing compositions provide hair styling benefits when applied to the hair, and when applied to the skin, barrier protection from surfactants or other materials having skin irritation potential.

### CROSS REFERENCE TO RELATED APPLICATION

This application claims the benefit of U.S. Provisional Application No. 60/325,021, filed Sep. 26, 2001.

### FIELD OF THE INVENTION

The present invention relates to personal cleansing compositions for rinse-off application and which comprise select silicone-resin containing adhesives. The compositions provide effective delivery and deposition of the silicone-resin containing adhesives as film-forming polymers onto hair, nails and skin.

### BACKGROUND OF THE INVENTION

There are many different personal cleansing compositions that have been described in the literature or otherwise available to consumers as commercial personal cleansing products. Such products include hair shampoos, body or hand cleansers, and other functionally similar products designed to topically cleanse various parts of the body.

Some of the personal cleansing compositions that are known or have otherwise been described in the literature contain various film forming polymers, including film-forming polymers in shampoo compositions that provide hair styling or conditioning benefits, and film-forming polymers in skin cleansing compositions to provide various cosmetic or active deposition benefits as well as skin conditioning benefits.

It has now been found that certain film-forming polymer materials can now be formulated into a personal cleansing composition to provide a variety of benefits. These film-forming materials are defined as those materials containing an organosiloxane resin in combination with an diorganosiloxane fluid, wherein the weight ratio of the organosiloxane resin to the diorganosiloxane fluid is from about 1:9 to about 10:1, or wherein the two materials are formulated into the composition as a silicone resin copolymer formed by the condensation of a organosiloxane resin and a diorganosiloxane fluid.

It has also been found that these new personal cleansing compositions, in addition to providing yet another personal cleansing composition not previously described in the art, also provide some unique benefits. When formulated into a shampoo composition, it has been found that the compositions provide hair styling benefits and hair volume reduction. When formulated into a skin cleansing composition, it has been found that the film-forming polymer readily deposits onto the skin even though it is formulated into a rinse-off cleansing composition, and helps eliminate or reduce skin irritation associated with the surfactant materials in the composition as well as other materials having similar skin irritation potential.

It is therefore and object of the present invention to provide an alternative personal cleansing composition not previously described or otherwise known in the various arts, and which provides improved delivery of a film-forming polymer to the hair, nails or skin. It is a further object of the present invention to provide a personal cleansing composition that provides a base composition that helps to eliminate or minimize skin irritation associated with the topical application of various surfactants and other materials having similar skin irritation potential.

### SUMMARY OF THE INVENTION

The present invention relates to personal cleansing compositions for topical rinse-off application to the hair, nails or skin. These compositions are rinse-off formulations that comprise from about 0.05% to about 40% by weight of a silicone resin-based adhesive, wherein the adhesive includes a silicone resin copolymer as a condensation product of a diorganosiloxane fluid and an organosiloxane resin; from about 0.5% to about 30% by weight of a cleansing surfactant; and from about 20% to about 99% by weight of a carrier liquid.

The present invention also relates to personal cleansing compositions for topical rinse-off application to the hair, nails or skin, said compositions comprising from about 0.05% to about 40% by weight of a silicone-resin based adhesive, wherein the adhesive is provided by a combination of an organosiloxane resin and an organosiloxane fluid, wherein the weight ratio of the organosiloxane resin to the diorganosiloxane fluid ranges from about 1:9 to about 10:1.

It has been found that the cleansing compositions of the present invention are effective in providing styling benefits when applied to and rinsed from the hair, including hair volume reduction and other hair cosmetic benefits made possible by the deposition onto hair of the film-forming materials as defined herein.

It is also believed that the personal cleansing compositions of the present invention provide adhesive and/or film-forming benefits when applied to the skin, even though the composition is a rinse-off cleansing composition. The film forming benefits are especially useful in protecting the skin from the irritation potential of different cleansing surfactants or other ingredients that tend to be irritating to the skin. It is believed that the compositions provide effective deposition of a cosmetically desirable film onto the skin during cleansing and subsequent rinsing, that provides protection of the skin from skin irritants within and external to the cleansing composition.

### DETAILED DESCRIPTION

The topical compositions of the present invention comprise a silicone-containing film forming material as defined herein, a cleansing surfactant, and a liquid carrier. These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water. The personal cleansing compositions of the present invention can be aqueous or anhydrous, but are preferably aqueous.

The term “volatile” as used herein, unless otherwise specified, refers to those materials having an average boiling point at one (1) atmosphere of pressure (atm) of less than about 250° C., more typically less than about 235° C. at one (1) atm.

The term “ambient conditions” as used herein, unless otherwise specified, refers to surrounding conditions at one atmosphere of pressure, 50% relative humidity, and 25° C.

The term “adhesive fluid” as used herein, unless otherwise specified, refers to the silicone-containing film forming materials formulated into the compositions of the present invention, wherein such materials are preferably solubilized in or otherwise in liquid form within the compositions. As such, the film-forming materials for use herein includes both solid and liquid materials, but when solid materials are used, the solid materials are preferably solubilized within the composition and subsequently maintained within the composition in liquid form, all under ambient conditions.

The term “personal cleansing composition” as used herein, unless otherwise specified, refers to the compositions of the present, wherein the compositions are intended to include only those compositions for topical application to the hair, nails or skin, and specifically excludes those compositions that are directed to other applications such as hard surface cleansing, fabric or laundry cleansing, and similar other applications not intended for topical application to the hair, nails or skin.

All viscosity values as described herein, unless otherwise specified, are expressed in terms of centistokes (cs) and are determined or otherwise measure by a Brookfield DV-II+ viscometer at 1 rpm at 25° C.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The personal cleansing compositions of the present invention can comprise, consist of, or consist essentially of the essential elements and limitations of the invention described herein, as well as any additional or optional ingredients, components, or limitations described herein or otherwise useful in personal cleansing compositions intended for topical application to the hair, nail or skin.

### Product Form

The personal cleansing compositions of the present invention can be formulated in any of a variety of product forms ranging from solids or semi-solids to liquids. The key to all of the varied product forms contemplated within the scope of the compositions of the present invention is the selected and defined combination of a film-forming material as defined herein, a liquid carrier, and a surfactant. These compositions include hair care products such as shampoos, skin cleansing or body wash compositions, hand cleansing products, and other functionally similar personal cleansing products.

All of the product forms contemplated for purposes of defining the present invention are rinse-off formulations, by which is meant the product is applied topically to the skin and then subsequently and immediately (i.e., within minutes) rinsed away with water, or otherwise wipe off using a substrate or other suitable removal means.

The compositions of the present invention are not intended for, and specifically exclude, leave-on formulations and applications.

### Surfactant

The personal cleansing compositions of the present invention comprise a cleansing surfactant suitable for application to the hair, nails or skin. Suitable surfactants for use herein include any known or otherwise effective cleansing surfactant suitable for application to the hair, nails or skin, and which is otherwise compatible with the other essential ingredients in the compositions.

Surfactants suitable for use in the personal cleansing compositions of the present invention include anionic, nonionic, cationic, zwitterionic or amphoteric surfactants, or combinations thereof, at product concentrations of from about 0.5% to about 30%, more typically from about 4% to about 30%, more typically from about 5% to about 25%, by weight, of the personal cleansing composition. Total surfactant concentrations in the personal cleansing compositions can vary depending on the type of surfactant selected, the presence of co-surfactants, the product results desired and so forth.

Anionic surfactants suitable for use in the personal cleansing compositions of the present invention include alkyl and alkyl ether sulfates. These materials have the respective formulae ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohol's having from about 8 to about 24 carbon atoms. Preferably, R has from about 10 to about 18 carbon atoms in both the alkyl and alkyl ether sulfates. The alcohol's can be derived from fats, e.g., coconut oil or tallow, or can be synthetic. Lauryl alcohol and straight chain alcohol's derived from coconut oil are preferred herein. Such alcohol's are reacted with about 1 to about 10, preferably from about 3 to about 5, and especially about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the personal cleansing compositions are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃—M], wherein R¹ is chosen from the group consisting of a straight or branched chain, saturated aliphatic hydrocarbon radical having from about 8 to about 24, preferably about 10 to about 18, carbon atoms; and M is a cation. Important examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Additional examples of suitable anionic surfactants are the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Other suitable anionic surfactants of this variety are described in U.S. Pat. Nos. 2,486,921, 2,486,922 and 2,396,278.

Still other suitable anionic surfactants are the succinamates, examples of which include disodium N-octadecylsulfosuccinamate; diammoniumlauryl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 12 to about 24 carbon atoms. The term “olefin sulfonates” is used herein to mean compounds which can be produced by the sulfonation of a-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form.

The a-olefins from which the olefin sulfonates are derived are mono-olefins having about 12 to about 24 carbon atoms, preferably about 14 to about 16 carbon atoms. Preferably, they are straight chain olefins.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

Another class of anionic surfactants suitable for use in the shampoo compositions are the b-alkyloxy alkane sulfonates. These compounds have the following formula:

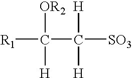

where R₁ is a straight chain alkyl group having from about 6 to about 20 carbon atoms, R₂ is a lower alkyl group having from about 1 (preferred) to about 3 carbon atoms, and M is a water-soluble cation as hereinbefore described.

Other suitable surfactants are described in McCutcheon's, Emulsifiers and Detergents, 1989 Annual, published by M. C. Publishing Co., and in U.S. Pat. No. 3,929,678, which descriptions are incorporated herein by reference.

Preferred anionic surfactants for use in the personal cleansing compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Amphoteric surfactants suitable for use in the personal cleansing compositions of the present invention includes those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Cationic surfactants can also be used in the personal cleansing compositions of the present invention, but are generally less preferred, and preferably represent less than about 5% by weight of the compositions.

Suitable nonionic surfactants for use in the personal cleansing compositions of the present invention include condensation products of alkylene oxide groups (hydrophilic in nature) with an organic hydrophobic compound, which may be aliphatic or alkyl aromatic in nature. Preferred classes of nonionic surfactants include:

1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the ethylene oxide being present in amounts equal to from about 10 to about 60 moles of ethylene oxide per mole of alkyl phenol;

2) nonionic surfactants derived from the condensation of ethylene oxide with the product resulting from the reaction of propylene oxide and ethylene diamine products;

3) condensation products of aliphatic alcohol's having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms;

4) long chain tertiary amine oxides corresponding to the following general formula:

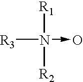

wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals;

5) long chain tertiary phosphine oxides corresponding to the following general formula:

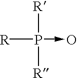

wherein R₁ contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms;

6) long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety;

7) alkyl polysaccharide (APS) surfactants such as the alkyl polyglycosides, as described in U.S. Pat. No. 4,565,647, which have a hydrophobic group with about 6 to about 30 carbon atoms and polysaccharide (e.g., polyglycoside) as the hydrophilic group, and optionally have a polyalkylene-oxide group joining the hydrophobic and hydrophilic moieties, wherein the alkyl group (i.e., the hydrophobic moiety) can be saturated or unsaturated, branched or unbranched, and unsubstituted or substituted (e.g., with hydroxy or cyclic rings); and

8) polyethylene glycol (PEG) glyceryl fatty esters, such as those of the formula R(O)OCH₂CH(OH)CH₂(OCH₂CH₂)ₙOH wherein n is from about 5 to about 200, preferably from about 20 to about 100, and R is an aliphatic hydrocarbyl having from about 8 to about 20 carbon atoms.

Zwitterionic surfactants suitable for use in the personal cleansing compositions include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

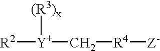

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use herein include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

### Liquid Carrier

The personal cleansing compositions of the present invention can be formulated in solid, semi-solid or liquid form, but must always comprise a liquid carrier suitable for topical application to the skin that is also compatible with the essential materials selected for use herein. The liquid carrier must be a liquid under ambient conditions or is otherwise in liquid form as formulated within the compositions, and helps to solubilize the film-forming material as described herein or otherwise helps to maintain the material as solubilized or in liquid form within the composition. The liquid carrier can be aqueous or anhydrous, and includes carrier liquids that are silicone-containing or non silicone-containing, volatile or non-volatile, aqueous or non-aqueous.

The personal cleansing compositions of the present invention preferably and typically comprise an aqueous carrier liquid, wherein the personal cleansing compositions preferably comprise water at a level that represents from 20% to about 98%, preferably from about 30% to about 90%, even more preferably from about 60% to about 90%, by weight of the personal cleansing compositions.

Other liquid carriers suitable for use herein, when used alone or in combination with water or other carrier liquid material, specifically excludes the organosiloxane fluids when such fluids are used in combination with an organosiloxane resin component of the composition. When the composition does not contain such silicone resin materials, then the organosiloxane fluid or other silicone containing fluids, including both volatile and non volatile silicones, are considered carrier liquids for purposes of more clearly defining the compositions of the present invention.

Volatile hydrocarbons suitable for use as a liquid carrier in the personal cleansing compositions herein include those hydrocarbons having boiling points in the range of from about 60° C. to about 260° C., more preferably volatile hydrocarbons having from about C₈ to about C₂₀ chain lengths, more preferably C₈ to C₂₀ isoparaffins. Preferred isoparaffins for use herein include isododecane, isohexadecane, isoeocosane, 2,2,4-trimethylpentane, 2,3-dimethylhexane and combinations thereof. Most preferred is isododecane.

Volatile silicones suitable for use as a liquid carrier in the personal cleansing compositions include those volatile silicones as described in Todd et al., “Volatile Silicone Fluids for Cosmetics”, Cosmetics and Toiletries, 91:27-32 (1976), which descriptions are incorporated herein by reference. The volatile silicone liquid can be linear, cyclic or branched, but is preferably a cyclic silicone having from about 3 to about 7, more preferably from about 4 to about 5, silicon atoms. Most preferably are those which conform to the formula:

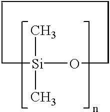

wherein n is from about 3 to about 7, preferably from about 4 to about 5, most preferably 5. Suitable volatile silicones for use herein include, but are not limited to, Cyclomethicone D-5 (commercially available from G. E. Silicones); Dow Coming 344, and Dow Corning 345 (commercially available from Dow Corning Corp.); GE 7207, GE 7158 and Silicone Fluids SF-1202 and SF-1 173 (available from General Electric Co.); SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof. Cyclopentasiloxane is most preferred among the volatile silicone liquids.

The liquid carrier for use in the compositions of the present invention can also include various other silicone-containing, volatile and non-volatile, diorganopolysiloxanes fluids that comprise repeating units corresponding to the formula (R₂SiO), where R is a monovalent hydrocarbon radical containing from 1 to 6 carbon atoms, preferably R is selected from methyl, ethyl, propyl, isopropyl, butyl, isobutyl, t-butyl, amyl, hexyl, vinyl, allyl, cyclohexyl, phenyl, fluoroalkyl and mixtures thereof. The fluid diorganopolysiloxane polymers may contain one or more of these hydrocarbon radicals as substituents on the siloxane polymer backbone. The diorganopolysiloxanes may be terminated by triorganosilyl groups of the formula (R′₃Si) where R′ is a radical selected from monovalent hydrocarbons containing from 1-6 carbon atoms, hydroxyl groups, alkoxyl groups and mixtures thereof. Polydimethylsiloxanes are preferred, especially linear dimethicones having a viscosity of from about 5cs to about 500,000 cs, preferably from about 10 cs to about 200,000 cs, as measured at 25° C.

Other liquid carriers suitable for use herein include non-volatile esters and other similar non-volatile fluids, non-limiting examples of which include isopropyl myristate, isopropyl palmitate, and combinations thereof.

### Silicone Resin Copolymer

The personal cleansing compositions of the present invention comprise a film-forming material that preferably comprises a silicone resin copolymer derived from the condensation or other functionally similar reaction or combination of an organosiloxane resin with a diorganopolysiloxane fluid. These silicone resin copolymers are known for use as adhesives in various consumers'products and applications, and are now formulated into the compositions of the present invention for the purpose of improving providing improved personal cleansing benefits as described herein.

It has been found that the these silicone resin copolymers as defined herein provide effective film-forming benefits even when formulated along with a cleansing surfactant in a rinse-off formulation

The concentration of silicone resin copolymer in the topical compositions of the present invention varies considerably depending upon other ingredients in the composition as well as the intended product form. Generally, silicone resin copolymer concentrations range from about 0.05% to about 40%, preferably from about 0.1% to about 30%, more preferably from about 2% to about 20%, by weight of the topical composition. The silicone resin copolymer is preferably further selected to have an average molecular weight of at least about 15,000, more preferably at least about 15,000 to 4 million, more preferably from about 100,000 to about 3 million, although it is understood that solid silicone resin copolymers can be solubilized and formulated into the composition, wherein such solid copolymers can have an immeasurably or almost immeasurably high viscosity.

The silicone resin copolymers can for use in the compositions can be prepared by any known or otherwise effective method or chemistry for making such materials, non limiting examples of which include co-hydrolysis or by reacting triorganosilanes or other similar siloxanes with a silica h. The silicone resin copolymers are generally prepared by mixing and heating together an organosiloxane resin, diorganosiloxane fluid, and catalyst, all as described herein, at a temperature of above about 1000, until the desired adhesive character of the resulting silicone resin copolymer is obtained. Mixing can be facilitated by the use of mutual solvents such as benzene, toluene, isododecane, xylene, naptha, mineral spirits or other suitable solvent, which is subsequently removed from the mixture during the heating and mixing process.

The silicone resin copolymers for use in the compositions of the present invention are preferably prepared by heating a mixture of (1) from about 45% to about 75% by weight of the organosiloxane resin as a condensation product of SiO₂ and R₃(SiO)_{0.5} units, wherein each R group is independently selected from methyl, ethyl, propyl or vinyl radicals, and the ratio of SiO₂ units to R₃(SiO)_{0.5} units in the organosiloxane resin is from about 0.6 to about 1.0; (2) from about 25% to about 55% by weight of a hydroxyl end blocked diorganopolysiloxane fluid having a viscosity in the range of from about 100 to about 1,000,000 cs at 25° C., wherein the organic substituents on the diorganosiloxane fluid are independently selected from methyl, ethyl, or vinyl radicals; and (3) from about 0.001% to about 5% by weight of a suitable catalyst, preferably an aliphatic organic amino compound selected from primary amines, secondary amines, tertiary amines, carboxylic acid salts of the above amines and quaternary ammonium salts. The mixture of materials is heated at a temperature above about 100° C. until the desired character of the resulting silicone resin copolymer is obtained.

Diorganosiloxane resins suitable for use herein are therefore those that preferably contain SiO₂ units and R₃(SiO)_{0.5} units (triorganosilyl) in a molecular ratio of from about 0.6 to about 1.0. Suitable triorganosilyl units for use in such diorganosiloxane resins include trimethylsilyl, triethylsilyl, methylmethylpropylsilyl, dimethylvinylsilyl, and combinations thereof. Preferred are trimethylsilyl units.

Diorganosiloxanes fluids suitable for use in preparing the silicone resin copolymers herein are hydroxyl end blocked diorganosiloxane polymers. The diorganosiloxanes are preferably linear polymers that contain only diorganosiloxane units, but can include small amounts of other materials such as triorganosiloxane units, monorganosiloxane units and SiO₂ units in minor amounts, typically less than about 1.0% by weight of the diorganosiloxane fluid, provided that the diorganosiloxane fluid remain hydroxyl end blocked to allow for the desired condensation reaction with the organosiloxane resin to form the silicone resin copolymer.

The organic substituents on the diorganosiloxane fluids for use in preparing the silicone resin copolymer can be any one or more of methyl, ethyl or vinyl radicals. Non-limiting examples of suitable diorganosiloxane fluids include ethylmethylpolysiloxane, copolymers of dimethylsiloxane and methylvinylsiloxane units, and mixtures of polymers or copolymers so long as such materials are hydroxyl end blocked. The viscosity of the diorganosiloxane polymer is preferably from about 100 to about 1,000,000 cs at 25° C.

The organic amino compound for use as a catalyst in preparing the silicone resin copolymer includes any aliphatic hydrocarbon amine; alkanol amine; carboxylic acid salt thereof; and tertiary amines such as trimethylamine, tributylamine, methyidiproppylamine, and quaternary ammonium salts. This includes primary amines such as hexylamine, butanolamine, and butylamine; secondary amines such as diethylamine, diethanolamine, ethylamylamine and propylhexylamine; tertiary amines such as trimethylamine, tirbutylamine, methyldipropylamine, tirpropanolamine, and methylpropylhexylamine; and quaternary ammonium salts such as tetramethylammonium acetate and methylethyldibutylammoniumchloride, including such as the quaternary ammonium emulsifying agents sold under various trade names, such as dioctadecyldimethylammonium chloride. In addition, any carboxylic acid salt of the amines, such as diethylamine acetate, butylamine octoate and trimethylamine laurate can be used. Tertiary amines are preferred, especially tertiary aliphatic amines.

Organosiloxane Resin Adhesives

The compositions of the present invention also preferably comprise an adhesive material that contains an organosiloxane resin, wherein the resin is used in combination with a liquid carrier component comprising a diorganopolysiloxane fluid. This combination of materials can be used alone or in combination with the above-described silicone resin copolymer.

The organosiloxane resin adhesive is preferably used in the composition of the present invention such that the weight ratio of the resin to the diorganopolysiloxane fluid is from about 1:9 to about 10:1, more preferably from about 1:1 to about 5:1, even more preferably from about 1:1 to about 3:1, and wherein the total concentration of the organosiloxane resin and the diorganopolysiloxane fluid ranges from about 1% to about 40%, more preferably from about 1% to about 30%, even more preferably from about 1% to about 20%, by weight of the composition.

The organosiloxane resin adhesive for use in the compositions of the present invention include combinations of R₃SiO_{1/2} (M units), R₂SiO (D units), RSiO_{3/2} (T units), SiO₂ (Q units) units in ratios to each other that satisfy the relationship RₙSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins are solids at about 25° C. and have a molecular weight range of from about 1,000 to about 10,000 grams/mole.

Preferred organosiloxane resins for use in the compositions of the present invention are those that contain repeating monofunctional or R₃SiO_{1/2} (M units) and quadrafunctional or SiO₂ (Q units), otherwise known as “MQ” resins as described in U.S. Pat. No. 5,330,747 (Krzysik), which descriptions are incorporated herein by reference. Examples of highly preferred organosiloxane resins are those in which the ratio of “M” to “Q” functional units is about 0.5 and the value of n is 1.5, non limiting examples of which are commercially available from Wacker Silicones Corporation of Adrian Mich. (e.g., Wacker 803 and 804) and the General Electric Company (e.g., G.E. 1170-002; G.E. SR1000).

Other suitable organosiloxane resins suitable for use herein include functionalized silicone resins, an example of which includes silicone ester waxes comprising moieties of the general formula:

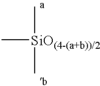

wherein R is an organic radical, R′ is a carboxylic acid ester, “a” and “b” are integers independently either 1 or 2 wherein a+b equals 2 or 3.

The organopolysiloxane fluid for use in combination with the organosiloxane resin described above can be any organopolysiloxane fluid that can be solubilized within the composition and that is otherwise compatible with the other essential ingredients of the composition. In this context, and for purposes of defining the compositions of the present invention, the organosiloxane fluid is considered part of the liquid carrier component of the compositions of the present invention. It is understood, however, that the organosiloxane fluid can still be used in the topical compositions even when the adhesive material does not contain an organosiloxane resin, and conversely, that the organosiloxane resin can still be used in the composition without the organosiloxane fluid, but that it is highly preferred that when such resin materials are used, they are used in combination with a organosiloxane fluid.

When used in combination with an organosiloxane resin, the concentration of the organopolysiloxane fluid in the topical composition of the present invention depends upon the total resin/fluid concentration and the relative weight ratios of the two, but will generally range from about 3% to about 15%, more typically from about 5% to about 10%, even more typically from about 5% to about 8%, by weight of the composition.

When used in combination with an organosiloxane resin, the organopolysiloxane fluid has a preferred viscosity of from about 100,000 to about 25,000,000 centistokes (cSt) at 25° C. and is preferably forms a solution with the organosiloxane resin and any other liquid carrier materials in the composition.

The diorganosiloxane fluid for use herein comprises repeating units that correspond to the formula (R₂SiO), where R is a monovalent hydrocarbon radical containing from 1 to 6 carbon atoms, preferably R is selected from methyl, ethyl, propyl, isopropyl, butyl, isobutyl, t-butyl, amyl, hexyl, vinyl, allyl, cyclohexyl, phenyl, fluoroalkyl and mixtures thereof. The organopolysiloxane fluid may contain one or more of these hydrocarbon radicals as substituents on the siloxane polymer backbone. The organopolysiloxane fluid may be terminated by triorganosilyl groups of the formula (R′₃Si) where R′ is a radical selected from monovalent hydrocarbons containing from 1-6 carbon atoms, hydroxyl groups, alkoxyl groups and mixtures thereof.

Non limiting examples of preferred organopolysiloxane fluids for use herein include poly(dimethylsiloxane) [PDMS] materials such as those available from General Electric as SE30, SE72, SE84, Viscasil®100M, Viscasil®300M, and Baysilone Fluid M 500,000.

### Optional Ingredients

The personal cleansing compositions of the present invention may further comprise other optional ingredients that may modify the physical, chemical, cosmetic or aesthetic characteristics of the compositions or serve as additional “active” components when deposited on the skin. The compositions may also further comprise optional inert ingredients. Many such optional ingredients are known for use in personal care compositions, and may also be used in the personal cleansing compositions herein, provided that such optional materials are compatible with the essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Non limiting examples of such optional ingredients include preservatives, deodorants, fragrances, deodorant perfumes, coloring agents or dyes, thickeners, sensates, sunscreens, gallants or other suspending agents, pH modifiers, co-solvents or other additional solvents, emollients, pharmaceutical actives, vitamins, and combinations thereof.

Other optional ingredients include silicone elastomer powders and fluids to provide any of a variety of product benefits, including improved product stability, application cosmetics, emolliency, and so forth. The concentration of the silicone elastomers in the composition preferably ranges from about 0.1% to about 20%, more preferably from about 0.5% to about 10%, by weight of the composition. In this context, the weight percentages are based upon the weight of the silicone elastomers material itself, excluding any silicone-containing fluid that typically accompanies such silicone elastomers materials in the formulation process. The silicone elastomers suitable for optional use herein include emulsifying and non-emulsifying silicone elastomers, non-limiting examples of which are described in U.S. Ser. No. 09/613,266 (assigned to The Procter & Gamble Company), which description is incorporated herein by reference.

Other optional ingredients include solid particulates that are and remain insoluble in the composition matrix, and include any inert or skin active solid particulate suitable for topical application to the hair, nails or skin. These insoluble solids optionally represent from about 0.1% to about 20%, by weight of the composition. Many of the other optional materials as described hereinabove can be selected and formulated within the composition as also insoluble solids. In this context, the term “insoluble” only means that all or most of the optional solid particulates remain as solid particulates within the finished composition and are not dissolved.

### Method of Use

The personal cleansing compositions of the present invention are applied topically to the desired area of the hair, skin or nails in an amount sufficient to provide effective delivery of the desired film-forming material to the applied surface. The compositions are preferably diluted with water prior to, during, or after topical application, and then subsequently rinsed or wiped off of the applied surface, preferably rinsed off of the applied surface using water or a water-insoluble substrate in combination with water.

The present invention is therefore directed to methods of cleansing the hair, nails or skin through the above-described application of the compositions of the present invention. The methods of the present invention are also directed to a method of providing effective delivery of the desired film-forming materials, and the resulting benefits from such effective delivery as described herein, to the applied surface through the above-described application of the compositions of the present invention.

### Method of Manufacture

The topical compositions of the present invention may be prepared by any known or otherwise effective technique, suitable for making and formulating the desired product form. Specific non-limiting examples of such methods as they are applied to specific embodiments of the present invention are described in the following examples.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, i.e., wt/wt percentages, unless otherwise specified.

The personal cleansing compositions described below provide deposition and adherence of the film-forming material onto the hair, skin or nails. When applied to the hair, the compositions provide hair styling and conditioning benefits, including hair volume reduction. When applied to the skin, or the skin and hair, the compositions help to prevent or inhibit skin irritation that would otherwise be associated with composition in the absence of the film-forming polymer component. Each of the exemplified compositions, therefore, provides improved film-forming benefits on the surfaces on which each is applied.

Personal Cleansing Compositions

The following examples described below are non-limiting examples of personal cleansing compositions of the present invention. These compositions can be applied to the hair, nail or skins as a rinse-off cleansing product.

The cleansing compositions of the present invention, including the exemplified shampoo embodiments described below, may be prepared using conventional formulation and mixing techniques. For example, the PSA is first to be dissolved in the C10-11 isoparrafin, and the PSA/C10-11 isoparrifin combination is then added to a premix of the surfactants, or some portion of the surfactants, and the solid components which had been heated to melt the solid components e.g., about 87° C. This mixture is then pumped through a high shear mill and cooled, and then the remaining components are mixed in. Alternatively, the PSA/C10-11 isoparrifin premix can be added to this final mix, after cooling. The composition has a final viscosity from about 2000 to about 12,000 cps. The viscosity of the composition can be adjusted using sodium chloride or ammonium xylenesulfonate as needed.

TABLE 1
Shampoo

| | Ex. | Ex. | Ex. | Ex. | Ex. |
|---|---|---|---|---|---|
| Ingredient | 1.1 | 1.2 | 1.3 | 1.4 | 1.5 |
| Ammonium Lauryl Sulfate | 3.0 | 3.0 | 3.0 | 3.0 | 3.0 |
| Ammonium Laureth Sulfate | 9.0 | 9.0 | 9.0 | 9.0 | 9.0 |
| Sodium Lauroamphoacetate | 5.1 | 5.1 | 5.1 | 5.1 | 5.1 |
| PSA 7-4400 (70% in | 4.3 | 1.4 | — | — | 2.8 |
| C10-11 isoparrifin) (1) | | | | | |
| PSA 7-4600 (70% in | — | — | — | 2.8 | — |
| C10-11 isoparrifin) (2) | | | | | |
| PSA 7-4500 (70% in | — | — | 2.8 | — | — |
| C10-11 isoparrifin) (3) | | | | | |
| Dihydrogenated Tallowamidoethyl | 1.0 | 1.0 | 1.0 | 1.0 | 1.0 |
| Hydroxyethylmonium Methosulfate | | | | | |
| (4) | | | | | |
| Citric Acid | 0.90 | 0.90 | 0.90 | 0.90 | 0.90 |
| PEG 14M | 0.30 | 0.30 | 0.30 | 0.30 | 0.30 |
| Trihydroxystearin | 0.15 | 0.15 | 0.15 | 0.15 | 0.15 |
| Glycol Distearate | 2.5 | 2.5 | 2.5 | 2.5 | 2.5 |
| C10-11 Isoparrifin | 1.2 | 1.2 | 1.2 | 1.2 | 1.2 |
| Fragrance | 0.85 | 0.85 | 0.85 | 0.85 | 0.85 |
| Cetyl Alcohol | 0.6 | 0.6 | 0.6 | 0.6 | 0.6 |
| Sodium Chloride | 0.45 | 0.45 | 0.45 | 0.45 | 0.45 |
| Guar Hydroxypropyl | 0.15 | 0.15 | 0.15 | 0.15 | 0.15 |
| Trimonium Chloride | | | | | |
| Polyquaternium 10 (JR30M) | — | — | 0.15 | — | — |
| Polyquaternium 10 (JR400) | — | 0.30 | — | — | 0.15 |
| Polyquaternium 10 (JR125) | 0.15 | — | — | 0.15 | — |
| Dimethicone | 0.25 | 0.25 | 0.25 | 0.25 | 0.25 |
| Panthenol | 0.05 | 0.05 | 0.05 | 0.05 | 0.05 |
| Pantyl Ethyl Ether | 0.05 | 0.05 | 0.05 | 0.05 | 0.05 |
| Methy Paraben | 0.20 | 0.20 | 0.20 | 0.20 | 0.20 |
| Methylchloroisothiazolinone, | 0.2 | 0.2 | 0.2 | 0.2 | 0.2 |
| Methylisothiazolinone | | | | | |
| Water | qs | qs | qs | qs | qs |
| | 100 | 100 | 100 | 100 | 100 |

(1) Available under the tradename PSA 7-4400 PSA Solids from Dow Corning Co. (USA)
(2) Available under the tradename PSA 7-4600 PSA Solids from Dow Corning Co. (USA)
(3) Available under the tradename PSA 7-4500 PSA Solids from Dow Corning Co. (USA)
(4) Available under the tradename Varisoft CB110 from Witco Chemical Co. (Dublin, Ohio, USA)

TABLE 2
Body Wash Compositions

| | Ex. | Ex. | Ex. | Ex. | Ex. | Ex. | Ex. | Ex. | Ex. | Ex. |
|---|---|---|---|---|---|---|---|---|---|---|
| Ingredient | 2.1 | 2.2 | 2.3 | 2.4 | 2.5 | 2.6 | 2.7 | 2.8 | 2.9 | 10 |
| Ammonium Laureth Sulfate | — | — | — | — | — | — | — | — | — | — |
| Ammonium Lauryl Sulfate | — | — | — | — | — | — | — | — | — | — |
| Sodium Lauroamphoacetate | — | — | — | — | — | — | — | — | — | — |
| Sodium Laureth Sulfate | 7.54 | 7.54 | 7.54 | 7.54 | 7.54 | 7.54 | 7.54 | 7.54 | 7.54 | 7.54 |
| Cocamidopropyl Betaine | 6.67 | 6.67 | 6.67 | 6.67 | 6.67 | 6.67 | 6.67 | 6.67 | 6.67 | 6.67 |
| Sodium Lauroyl Sarcosinate | 0.65 | 0.65 | 0.65 | 0.65 | 0.65 | 0.65 | 0.65 | 0.65 | 0.65 | 0.65 |
| Citric Acid | 0.26 | 0.26 | 0.26 | 0.26 | 0.26 | 0.26 | 0.26 | 0.26 | 0.26 | 0.26 |
| Trihydroxystearin | 1.00 | 1.00 | 1.00 | 1.00 | 1.00 | 1.00 | 1.00 | 1.00 | 1.00 | 1.00 |
| Starch Copolymer | 0.50 | 0.50 | 0.50 | 0.50 | 0.50 | 0.50 | 0.50 | 0.50 | 0.50 | 0.50 |
| Guar | 0.50 | 0.50 | 0.50 | 0.50 | 0.50 | 0.50 | 0.50 | 0.50 | 0.50 | 0.50 |
| Hydroxypropyltrimonium | | | | | | | | | | |
| Chloride | | | | | | | | | | |
| Lauryl Alcohol | 0.65 | 0.65 | 0.65 | 0.65 | 0.65 | 0.65 | 0.65 | 0.65 | 0.65 | 0.65 |
| PSA 7-4100/Trimethylated | 7.14 | 28.57 | 14.29 | — | — | — | — | — | — | — |
| silica treated with dimethyl | | | | | | | | | | |
| siloxane (70% in isododecane) | | | | | | | | | | |
| (1) | | | | | | | | | | |
| PSA 7-4100/Trimethylated | — | — | — | 25.00 | — | — | — | — | — | — |
| silica treated with dimethyl | | | | | | | | | | |
| siloxane (40% in isododecane) | | | | | | | | | | |
| (1) | | | | | | | | | | |
| PSA 7-4100/Trimethylated | — | — | — | — | 20 | — | — | — | — | — |
| silica treated with dimethyl | | | | | | | | | | |
| siloxane (50% in isopropyl | | | | | | | | | | |
| myristate) (1) | | | | | | | | | | |
| PSA 7-4200/Trimethylated | — | — | — | — | — | 14.29 | — | — | — | — |
| silica treated with dimethyl | | | | | | | | | | |
| siloxane (70% in isododecane) | | | | | | | | | | |
| (2) | | | | | | | | | | |
| PSA 7-4300/Trimethylated | — | — | — | — | — | — | 14.29 | — | — | — |
| silica treated with dimethyl | | | | | | | | | | |
| siloxane (70% in isododecane) | | | | | | | | | | |
| (3) | | | | | | | | | | |
| PSA 7-4400/Trimethylated | — | — | — | — | — | — | — | 14.29 | — | |
| silica treated with dimethyl | | | | | | | | | | |
| siloxane (70% in isododecane) | | | | | | | | | | |
| (4) | | | | | | | | | | |
| PSA 7-4500/Trimethylated | — | — | — | — | — | — | — | — | 14.29 | — |
| silica treated with dimethyl | | | | | | | | | | |
| siloxane (70% in isododecane) | | | | | | | | | | |
| (5) | | | | | | | | | | |
| PSA 7-4600/Trimethylated | — | — | — | — | — | — | — | — | — | 14.29 |
| silica treated with dimethyl | | | | | | | | | | |
| siloxane (70% in isododecane) | | | | | | | | | | |
| (6) | | | | | | | | | | |
| MQ SR1000 | — | — | — | — | — | — | — | — | — | — |
| Resin/Trimethylsiloxysilicate | | | | | | | | | | |
| (75% in isododecane) (7) | | | | | | | | | | |
| SE30 Gum/Dimethicone | — | — | — | — | — | — | — | — | — | — |
| (40% in isododecane) (8) | | | | | | | | | | |
| DMDM Hydantoin | 0.21 | 0.21 | 0.21 | 0.21 | 0.21 | 0.21 | 0.21 | 0.21 | 0.21 | 0.21 |
| Sodium Benzoate | 0.25 | 0.25 | 0.25 | 0.25 | 0.25 | 0.25 | 0.25 | 0.25 | 0.25 | 0.25 |
| Disodium EDTA | 0.10 | 0.10 | 0.10 | 0.10 | 0.10 | 0.10 | 0.10 | 0.10 | 0.10 | 0.10 |
| Water | qs | qs | qs | qs | qs | qs | qs | qs | qs | qs |
| | 100 | 100 | 100 | 100 | 100 | 100 | 100 | 100 | 100 | 100 |

(1) Available under the tradename PSA 7-4100 Solids from Dow corning Co. (USA)
(2) Available under the tradename PSA 7-4200 Solids from Dow corning Co. (USA)
(3) Available under the tradename PSA 7-4300 Solids from Dow corning Co. (USA)
(4) Available under the tradename PSA 7-4400 Solids from Dow corning Co. (USA)
(5) Available under the tradename PSA 7-4500 Solids from Dow corning Co. (USA)
(6) Available under the tradename PSA 7-4600 Solids from Dow corning Co. (USA)
(7) Available under the tradename MQ SR1000 Resin from GE Silicones (USA)
(8) Available under the tradename SE30 Silicone Gum from GE Silicones (USA)

TABLE 3
Body Wash Compositions

| | Ex. | Ex. | Ex. |
|---|---|---|---|
| Ingredient | 3.1 | 3.2 | 3.3 |
| Ammonium Laureth Sulfate | 3.00 | — | — |
| Ammonium Lauryl Sulfate | 4.00 | — | — |
| Sodium Lauroamphoacetate | 4.00 | — | — |
| Sodium Laureth Sulfate | — | 7.54 | 7.54 |
| Cocamidopropyl Betaine | — | 6.67 | 6.67 |
| Sodium Lauroyl Sarcosinate | 4.50 | 0.65 | 0.65 |
| Citric Acid | 0.26 | 0.26 | 0.26 |
| Trihydroxystearin | 1.00 | 1.00 | 1.00 |
| Starch Copolymer | 0.50 | 0.50 | 0.50 |
| Guar | 0.50 | 0.50 | 0.50 |
| Hydroxypropyltrimonium | | | |
| Chloride | | | |
| Lauryl Alcohol | 0.65 | 0.65 | 0.65 |
| PSA 7-4100/Trimethylated | 14.29 | 28.57 | 14.29 |
| silica treated with dimethyl | | | |
| siloxane (70% in isododecane) | | | |
| (1) | | | |
| PSA 7-4100/Trimethylated | — | — | — |
| silica treated with dimethyl | | | |
| siloxane (40% in isododecane) | | | |
| (1) | | | |
| PSA 7-4100/Trimethylated | — | — | — |
| silica treated with dimethyl | | | |
| siloxane (50% in isopropyl | | | |
| myristate) (1) | | | |
| PSA 7-4200/Trimethylated | — | — | — |
| silica treated with dimethyl | | | |
| siloxane (70% in isododecane) | | | |
| (2) | | | |
| PSA 7-4300/Trimethylated | — | — | — |
| silica treated with dimethyl | | | |
| siloxane (70% in isododecane) | | | |
| (3) | | | |
| PSA 7-4400/Trimethylated | — | — | — |
| silica treated with dimethyl | | | |
| siloxane (70% in isododecane) | | | |
| (4) | | | |
| PSA 7-4500/Trimethylated | — | — | — |
| silica treated with dimethyl | | | |
| siloxane (70% in isododecane) | | | |
| (5) | | | |
| PSA 7-4600/Trimethylated | — | — | — |
| silica treated with dimethyl | | | |
| siloxane (70% in isododecane) | | | |
| (6) | | | |
| MQ SR1000 | — | 13.33 | 8.67 |
| Resin/Trimethylsiloxysilicate | | | |
| (75% in isododecane) (7) | | | |
| SE30 Gum/Dimethicone (40% | — | — | 8.75 |
| in isododecane) (8) | | | |
| DMDM Hydantoin | 0.21 | 0.21 | 0.21 |
| Sodium Benzoate | 0.25 | 0.25 | 0.25 |
| Disodium EDTA | 0.10 | 0.10 | 0.10 |
| Water | qs | qs | qs |
| | 100 | 100 | 100 |

(1) Available under the tradename PSA 7-4100 Solids from Dow corning Co. (USA)
(2) Available under the tradename PSA 7-4200 Solids from Dow corning Co. (USA)
(3) Available under the tradename PSA 7-4300 Solids from Dow corning Co. (USA)
(4) Available under the tradename PSA 7-4400 Solids from Dow corning Co. (USA)
(5) Available under the tradename PSA 7-4500 Solids from Dow corning Co. (USA)
(6) Available under the tradename PSA 7-4600 Solids from Dow corning Co. (USA)
(7) Available under the tradename MQ SR1000 Resin from GE Silicones (USA)
(8) Available under the tradename SE30 Silicone Gum from GE Silicones (USA)

What is claimed is:

1. Personal cleansing compositions for rinse-off application to the hair or skin, said compositions comprising:
(A) from about 0.1% to about 30% by weight of a silicone resin copolymer as a condensation product of a diorganosiloxane fluid and an organosiloxane resin;
(B) from about 0.5% to about 30% by weight of a cleansing surfactant; and
(C) from about 20% to about 98.9% by weight of a carrier liquid;
wherein the personal cleansing composition is a rinse-off composition for application to the hair or skin.

2. The personal cleansing composition of claim 1 wherein the silicone resin copolymer has an average molecular weight of at least about 15,000.

3. The personal cleansing composition of claim 1 wherein the silicone resin copolymer has an average molecular weight of from about 100,000 to about 3 million.

4. The personal cleansing composition of claim 1 wherein the diorganosiloxane fluid is a hydroxyl end blocked diorganopolysiloxane having a viscosity of from about 100 to about 1,000,000 cs at 25° C., wherein the organic substituents on the diorganopolysiloxane fluid are selected from the group consisting of methyl, ethyl, and vinyl radicals.

5. The personal cleansing composition of claim 4 wherein the organosiloxane resin is as a condensation product of SiO₂ and R₃(SiO)_{0.5} units; wherein each R group is independently selected from the group consisting of methyl, ethyl, propyl and vinyl radicals; and the molar ratio of SiO₂ units to R₃(SiO)_{0.5} units in the silicone resin is from about 0.6 to about 1.0.

6. The personal cleansing composition of claim 1 wherein the carrier liquid is an aqueous liquid and the composition comprises from about 20% to about 98% by weight of water.

7. The personal cleansing composition of claim 1 wherein the composition contains from about 30% to about 90% by weight of water.

8. The personal cleansing composition of claim 1, wherein the composition further comprises from about 0.1% to about 20% by weight of solid particulates.

9. Personal cleansing compositions for application to the hair, nails or skin, said compositions comprising:
(A) from about 0.1% to about 30% by weight of a film-forming material comprising an organosiloxane resin and a diorganosiloxane fluid at a weight ratio of said resin to said fluid of from about 1:9 to about 10:1.
(B) from about 0.5% to about 30% by weight of a cleansing surfactant; and
(C) from about 20% to about 99% by weight of a carrier liquid;
wherein the personal cleansing composition is a rinse-off composition for application to the hair or skin.

10. The personal cleansing composition of claim 9 wherein the weight ratio of the organosiloxane resin to the diorganosilxane fluid is from about 1:1 to about 3:1

11. The personal cleansing composition of claim 9 wherein the diorganosiloxane fluid has a viscosity of from about 100 to about 1,000,000 cs at 25° C. and the organic substituents on the diorganopolysiloxane fluid are selected from the group consisting of methyl, ethyl, and vinyl radicals.

12. The personal cleansing composition of claim 9 wherein the organosiloxane resin is a condensation product of SiO₂ and R₃(SiO)_{0.5} units; wherein each R group is independently selected from the group consisting of methyl, ethyl, propyl and vinyl radicals; and the molar ratio of SiO₂ units to R₃(SiO)_{0.5} units in the silicone resin is from about 0.6 to about 1.0.

13. The personal cleansing composition of claim 9 wherein the organosiloxane resin represents from about 1% to about 10% by weight of the composition, and the diorganosiloxane fluid represents from about 1% to about 10% by weight of the composition.

14. The personal cleansing composition of claim 9 wherein the carrier liquid is an aqueous liquid and the composition comprises from about 20% to about 98% by weight of water.

15. The personal cleansing composition of claim 9 wherein the composition contains from about 30% to about 90% by weight of water.

16. The personal cleansing composition of claim 9, wherein the composition further comprises from about 0.1% to about 20% by weight of solid particulates.

17. A method of cleansing the skin, said method comprising the topical application of the composition of claim 1 to the skin followed by rinsing of the applied area of the skin with water.

18. A method of cleansing the skin, said method comprising the topical application of the composition of claim 9 to the skin followed by rinsing of the applied area of the skin with water.
